(19) United States
(12) Patent Application Publication
(10) Pub. No.: US 2024/0299073 A1
(43) Pub. Date: Sep. 12, 2024

(54) MONOPOLAR AND BIPOLAR ELECTROPORATION CATHETER

(71) Applicants: CENTRE HOSPITALIER UNIVERSITAIRE DE BORDEAUX, TALENCE (FR); UNIVERSITE DE BORDEAUX, BORDEAUX (FR); FONDATION BORDEAUX UNIVERSITE, BORDEAUX (FR)

(72) Inventors: Pierre JAIS, SAINT MEDARD EN JALLES (FR); Rémi DUBOIS, MÉRIGNAC (FR)

(21) Appl. No.: 18/259,026

(22) PCT Filed: Dec. 21, 2021
(86) PCT No.: PCT/EP2021/087144

§ 371 Date: Jun. 22, 2023

(30) Foreign Application Priority Data
Dec. 23, 2020 (FR) FR2014047

(51) Int. Cl. A61B 18/00

(52) U.S. Cl. CPC A61B 18/00; A61B 2018/00077; A61B 2018/00083; A61B 2018/00172; A61B 2018/00184; A61B 2018/00267; A61B 2018/00375; A61B 2018/00577; A61B 2018/00702; A61B 2018/00839

(57) ABSTRACT

A medical device includes a catheter including a movable distal portion including a body evolving longitudinally within a first lumen and a plurality of branches, a distal end of each branch including an electrically active zone and being movable between a retracted position and a deployed position, the medical device being electrically configurable to deliver a first quantity of electrical energy to a first set of electrodes in the deployed position and a second quantity of electrical energy to a second set of electrodes in the retracted position.

### FIELD OF THE INVENTION

The field of the invention relates to the field of catheters dedicated to atrial fibrillation treatment. The field of the invention relates in particular to the field of treatment of this pathology by isolation of the pulmonary vein by electroporation implemented using catheters comprising one or more electrodes.

### PRIOR ART

Today, atrial fibrillation is a very common cardiac disorder. Atrial fibrillation (AF) is an arrhythmia defined by chaotic activation of the atria. It is triggered by atrial extrasystoles initiating multiple and variable reentry. Pulmonary veins, a source of extrasystoles and a substrate for reentry, are recognized as the basic structures for initiating and maintaining atrial fibrillation. They are therefore the primary target of ablation for the treatment of paroxysmal atrial fibrillation. When arrhythmia has reached a more advanced stage, fibrillation is persistent, and in addition to treatment on the pulmonary veins, linear lesions may also have to be delivered.

In particular, ablation of the pulmonary veins PVI (Pulmonary Vein Isolation) may be performed using an electroporation method. Pulsed Field Electroporation (PFE) ablation is a method wherein a strong, pulsed electrical field is applied to create pores in the membranes of cardiac cells. These pores will cause the death of these targeted cells (irreversible electroporation) and stop the chaotic activation of the atria. The advantage of this method is that it does not result in a thermal rise, or a very low thermal rise, of a magnitude of a degree Celsius, in the targeted tissues and in adjacent tissues. This method also has some degree of tissue selectivity. This is because cardiac cells are much more sensitive than those of adjacent structures.

There are two main modes of electroporation delivery, bipolar and unipolar.

During bipolar electroporation ablation, a catheter with a plurality of electrodes is used, which is inserted into the pulmonary vein. This catheter is connected to a generator that generates voltage between two catheter electrodes, resulting in an electrical field. It is this electrical field that will create pores in the cells that make up the tissue of the pulmonary vein. It is in particular known in document US20180085160 a basket-shaped catheter comprising five branches, each carrying 4 electrodes. When the catheter is deployed, the branches move away from each other and the electrodes are brought closer to the walls of the vein to be treated. In the deployed position, treatment of the entire pulmonary vein is performed, while the retracted position allows navigation of the catheter into the atria and then into the pulmonary veins.

It may also be useful to perform linear lesions in the atria, especially in patients with persistent atrial fibrillations. In this case, after removal of the large-diameter basket electroporation catheter, a new smaller-diameter electroporation catheter is introduced to achieve a linear lesion on the atrium wall. It is a basket-shaped catheter that creates smaller lesions than the catheter dedicated to pulmonary veins. By deploying them sequentially and coalescing, a linear lesion may be formed, or focal targets may be treated.

Electroporation may also be delivered in unipolar mode. This involves applying a pulsed electrical field between two electrodes, one of which is located on the catheter in contact with the cardiac target, and the other, larger, is located outside the body of the patient, most often attached to the chest. Both unipolar and bipolar modes may be used for isolation of pulmonary veins or for linear lesions. They each have advantages and disadvantages.

One disadvantage of these modes of application of electroporation when treating a patient who would require isolation of the pulmonary veins and completion of linear lesions is that it is necessary to use two ablation catheters. A first part of the procedure involves performing a circular lesion around the ostium of the pulmonary veins, then performing one or more linear lesions with another catheter. To perform these two manipulations, it is usually necessary to remove the electroporation catheter dedicated to the pulmonary veins, then to insert a new catheter into the body of the patient that is suitable for performing linear electroporation. This succession of catheter insertion poses several problems, firstly the risk to the patient of introducing a new catheter, secondly the duration of the procedure involved in removing and inserting a new catheter, and above all the very high added cost of each of these catheters.

### SUMMARY OF THE INVENTION

The medical device according to the invention makes it possible to overcome the aforementioned problems.

According to one aspect, the invention relates to a medical device comprising a catheter comprising a movable distal portion comprising a body evolving longitudinally within a first lumen and a plurality of branches, a distal end of each branch being fastened along a perimeter of said body and a proximal end being fastened along a perimeter of said first lumen, each branch having a face having at least one electrically active zone forming an electrode. The branches are movable between at least two positions:
a retracted position wherein the branches are each arranged along an axis parallel to the longitudinal axis of the body;
a deployed position wherein the branches each form an arc, each arc extending along a plane to which the longitudinal axis of the body belongs.

The medical device is electrically configurable to deliver a first quantity of electrical energy to a first set of electrodes in the deployed position and a second quantity of electrical energy to a second set of electrodes in the retracted position.

The medical device according to the invention makes it possible, with the same catheter, to perform circular lesions around the pulmonary veins in a deployed mode, and to perform linear lesions with the same catheter in a retracted position of the catheter.

This arrangement makes it possible to use the same catheter during a pulmonary vein isolation procedure that also requires linear lesions, thus saving time during the procedure, reducing the risk of complications for the patient, and finally significantly reducing the material cost of the procedure, as the unit cost of a catheter is very high. Of course, this arrangement allows only the pulmonary veins to be treated if necessary.

According to one aspect, the body of the distal portion comprises a second lumen designed to accommodate a catheter guiding device. This characteristic allows guiding the catheter into the blood vessels of a patient to place the distal portion into the pulmonary vein.

According to one aspect, the medical device is configured to deliver the first quantity of energy or the second quantity of energy according to a train of electrical pulses generated within at least one branch causing the creation of electrical voltages between two electrodes. This characteristic allows efficient electroporation thanks to the electrical pulse train.

According to one aspect, a first electrode of the medical device is at least one first branch and the second electrode is at least one second branch. This arrangement gives the device the advantage of being able to apply an electrical voltage between two of its branches to perform bipolar electroporation.

According to one aspect, the first quantity of electrical energy is delivered according to a sequence comprising a succession of power supplies of electrode pairs. The advantage of this characteristic is to successively solicit different electrode pairs to effectively enable electroporation of the entire perimeter of the pulmonary vein.

According to one aspect, a first electrode is at least one branch and the second electrode is an electrode external to the catheter. This arrangement makes it possible to perform electroporation in a unipolar mode, with an external electrode placed on the body of the patient, to perform linear lesions or to treat the pulmonary vein.

According to one aspect, the first electrode is formed by several branches and the second electrode is an electrode external to the catheter. This arrangement allows electroporation in a unipolar mode using several electrodes formed by several branches as the first electrode.

According to one aspect, each branch comprises a core of electrically conductive material surrounded by an electrically insulating sheath comprising an opening, the active zone being arranged on a face opposite a longitudinal axis of the body through said opening. This arrangement offers an easy-to-implement branch architecture allowing electrical insulation of the branch from active zones.

According to one aspect, the branch is made of a shape memory material. This characteristic makes it possible to have a branch with a superelasticity property that may deform when it moves from the retracted position to the deployed position (and vice versa) without entering its field of plastic deformations. “Superelasticity” means the ability of a material to deform strongly and return to its original position, reversibly.

According to one aspect, the conductive material is Nitinol. Nitinol is a memory alloy that has a superelasticity property.

According to one aspect, at least one electrode of a branch comprises a printed circuit on an outer layer of the branch. This arrangement makes it possible to place an electrode made by the printed circuit board where desired on the branch.

According to one aspect, at least one branch comprises at least one measuring electrode capable of recording an electrical activity. This arrangement allows electrical measurements to be taken from the measuring electrode to, for example, facilitate and monitor proper placement of the catheter in the pulmonary vein or monitor the isolation thereof.

According to one aspect, each electrically active zone forms a configurable electrode to deliver a quantity of energy and be able to record an electrical activity. According to this characteristic, the electrodes formed by the active zones are used to perform electroporation by delivering energy and also serve as a measuring electrode to measure electrical activity.

According to one aspect, at least one branch comprises several distinct electrically active zones, each electrically active zone forming an electrode, all the electrodes of the branches being configurable to deliver a quantity of energy and/or being able to record an electrical activity. Thus, the electrodes formed by the active zones are used to perform electroporation by delivering energy and at the same time serve as a measuring electrode to measure an electrical activity.

According to one aspect, each branch comprises at least two electrically active zones each forming a conductive part of an electrical circuit of the medical device, each of the electrical circuits being independent of the other to propagate respectively a measured electrical current and a generated electrical current.

According to one aspect, each branch comprises at least one electrically active zone forming a conductive part of a single electrical circuit of the medical device to propagate a measured electrical current or generated electrical current.

According to one aspect, the body of the distal portion of the catheter comprises a measuring electrode capable of recording electrical activity, preferably in conjunction with an electrode of one of the branches. This characteristic makes it possible to measure an electrical voltage between a branch and the body of the distal portion.

According to one aspect, each branch comprises two lateral edges, each lateral edge comprising an electrical insulation electrically insulating said branch from a branch located in the vicinity of the latter. This arrangement offers the advantage of electrically and reliably insulating a branch of the branches in the vicinity of the latter, in order to avoid the formation of an electrical contact between the latter, and thus prevent a possible short circuit between two branches. This arrangement also makes it possible to electrically insulate a branch of the branches in the vicinity of the latter when the catheter is in the retracted position.

According to one aspect, each active zone of each branch is located on a distant portion of its distal end and its proximal end, the active zone preferably being at least 7 millimeters away from the distal end.

According to one aspect, an active zone of the distal end of a branch is longitudinally offset from an active zone of the distal end of a consecutive branch on the perimeter of the first lumen. This characteristic gives the advantage of allowing a good distribution of the active zones along a perimeter of the pulmonary vein, in order to be able to perform electroporation isolation of the latter even when the catheter is, in its deployed position, slightly askew in said pulmonary vein.

According to one aspect, the branches have a general ribbon shape. The ribbon shape allows good stability of the branch when the catheter is in the deployed position, with the faces bearing on the walls of the pulmonary vein, or the ostium of the pulmonary vein. This characteristic makes it possible to limit unwanted torsion of the branches. In the retracted position, the ribbon shape allows for an arrangement in rows of the branches around the body, which facilitates linear electroporation when the catheter is in the retracted position.

According to one aspect, the fastening of the branches along the perimeter of the first lumen is carried out via a fastening ring to which the proximal ends are fastened, the fastening ring itself being linked to the perimeter of the first lumen. This characteristic allows the quantity of travel to be adjusted between the retracted and deployed positions to best suit the size of the pulmonary vein.

### BRIEF DESCRIPTION OF THE FIGURES

Further characteristics and advantages of the invention will become clearer upon reading the following detailed description, in reference to the appended figures, that show:

FIG. 1: a profile view of a medical device according to the invention, in a deployed position.

FIG. 2: a profile view of the device of FIG. 1, in a retracted position.

FIG. 3: a top view of the medical device of FIG. 1, in its deployed position.

FIG. 4: a top view of the medical device of FIG. 1, in its retracted position.

FIG. 5: a profile view of a medical device according to a second embodiment of the invention.

FIG. 6: a sectional view of a branch of a catheter according to the invention.

FIG. 7: a sectional view of a branch of a catheter comprising a printed circuit.

FIG. 8: a front view of two successive branches of the medical device according to a third embodiment of the invention

FIG. 9: a partial profile view of a distal portion of the medical device of FIG. 8 having two successive branches according to one embodiment of the invention.

### DESCRIPTION OF THE INVENTION

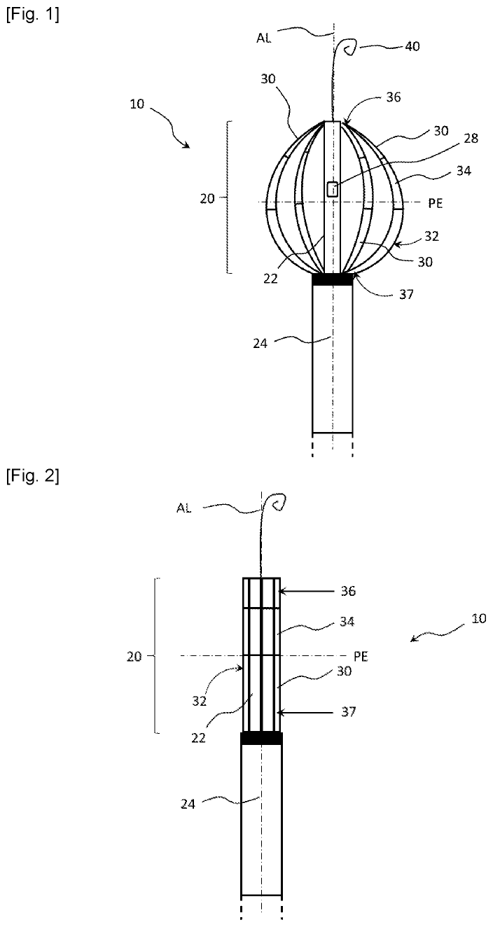

FIG. 1 shows an embodiment of a medical device according to the invention, shown in a lateral view. The medical device comprises a catheter 10 comprising a distal portion 20.

### The Catheter

Distal portion 20 is defined as a portion of the catheter 10 located at its distal end, i.e., on the end that is going to be located at the front portion of the catheter 10, the portion that is first introduced into the body of the patient.

The distal portion 20 comprises a movable body 22 on the catheter 10. The body 22 is movable in a longitudinal direction of the catheter. This direction is given by a longitudinal axis AL of the body 22. The movement of the body 22 may occur through a first lumen 24 of the catheter 10. “First lumen” 24 means a cavity arranged in the catheter in the vicinity of the distal portion 20, this cavity having the shape of a cylindrical bore in the catheter. The body 22 of the distal portion 20 thus extends partially into this lumen 24, and may slide inside it in a longitudinal movement. In other words, the body 22 of the distal portion 20 is, in a retracted position, almost entirely out of the lumen 24 (as shown in FIG. 2), and may in the deployed position be almost entirely inside the lumen 24, with only its distal end flush with the lumen 24.

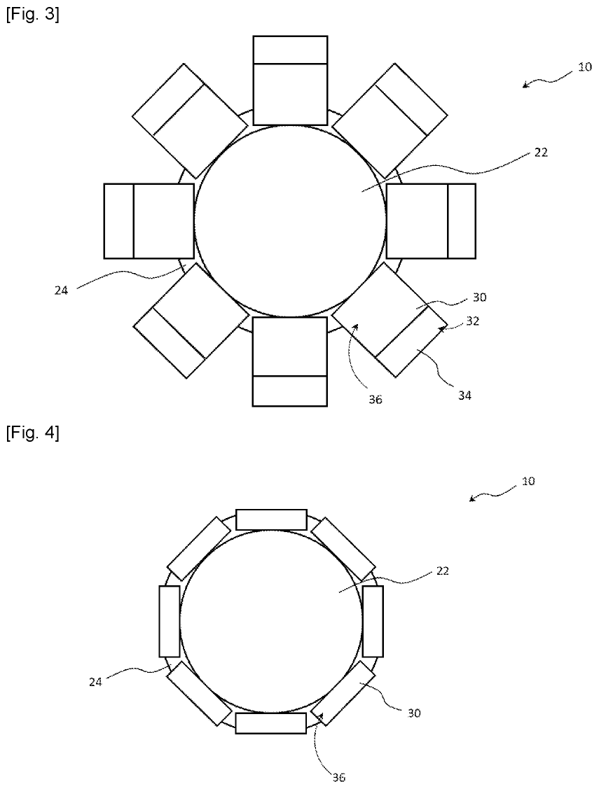

FIGS. 3 and 4 show the catheter 10 in the deployed and retracted position respectively, each shown in the top view, i.e., with the view that the distal portion 20 of the catheter 10 is shown in the foreground, the rest of the catheter 10 being shown in the background.

The distal portion 20 of the catheter comprises a plurality of branches 30 fastened to said portion. Each branch 30 comprises a distal end 36 that is fastened to the body 22. More precisely, the distal end 36 of each branch 30 is fastened on a perimeter of the body 22, i.e. on an external surface of the body 22. Each branch 30 comprises a proximal end 37 that is fastened to the catheter along a perimeter of the first lumen 24. The proximal ends 37 may be fastened directly to the perimeter of a through-end of the first lumen 24, directly in the vicinity of the body 22. The proximal ends 37 may also be fastened to a perimeter of the first lumen 34 on an external surface of the catheter 10.

The catheter 10 of FIG. 1 comprises eight branches 30, of which only the four located towards the foreground are visible in FIG. 1. However, a wide variety of numbers of branches 30 may be considered, for example two, three, four, five, six, seven or nine. A number of branches of up to fifteen, or even more, may be considered.

Advantageously, the branches 30 are arranged uniformly along the perimeter of the body 22. In other terms, the branches 30 have a regular angular arrangement around the body 22. This characteristic makes it easier to deliver the desired quantity of energy, as the distance between each branch is equal.

Each branch comprises a face 32 which comprises at least one electrically active zone 34 forming an electrode. This electrically active zone 34, located on a surface of branch 30, is ideally made of conductive material and is suitable as an electrode.

The arms 30 are movable between a deployed position and a retracted position.

In the retracted position, shown in FIG. 2, the branches 30 are straight and arranged along the body 22, on its perimeter. Each branch 30 is arranged along an axis parallel to the longitudinal axis AL of the body 22. Thus, in the retracted position, the branches extend in the continuity of the catheter around the body 22 of the distal portion 20.

In the deployed position, each branch 30 forms an arc extending between its distal end 36 and its proximal end 37. The arc formed by each branch 30 extends along a plane to which the longitudinal axis AL of the body 22 belongs. In other words, each branch 30, in the deployed position, extends in a plane to which the longitudinal axis AL of the body 22 of the distal portion 20 belongs. This arrangement increases the stability of the branches 30 during deployment. Indeed, a deployment in another plane would require a torsion of said branches 30, which would increase instability during deployment. In this way, the invention makes it possible to obtain a geometry of the branches in the deployed position which is regular.

The medical device according to the invention is configurable to electrically deliver a first quantity of electrical energy to a first set of electrodes in a deployed position. Thus, when the catheter is in the deployed position, it is adapted to deliver a voltage between two electrodes of active zones 34 of two different branches 30.

The device according to the invention is also configurable to electrically deliver a second quantity of energy to a second set of electrodes in the retracted position. Thus, when the catheter is in the retracted position, it is possible to apply an electrical potential to at least one of the electrodes, in order to deliver the second quantity of energy.

This configuration of the medical device according to the invention makes it possible to deliver a quantity of electrical energy both when the catheter 10 is in the deployed position and when it is in the retracted position.

This arrangement therefore has the advantage of allowing the catheter 10 to be used to perform pulmonary vein isolation by electroporation (unipolar or bipolar) when in the deployed position, and, with the same catheter, to perform a linear lesion when the catheter 10 is in the retracted position, possibly in unipolar mode.

The medical device according to the invention therefore makes it possible to avoid having to use two different catheters to perform a circular lesion and a linear lesion, which makes it possible to facilitate the atrial fibrillation treatment procedure by avoiding the need to remove a first catheter capable of performing isolation of the pulmonary veins, then insert a second catheter capable of performing a linear electroporation. This makes the procedure faster and less risky for the patient. Furthermore, the invention makes it possible to reduce the cost of such an operation by reducing the number of catheters used, which are very costly.

To pass the catheter 10 from the retracted position to the deployed position, the body 22 is slid into the lumen 24 in a direction from its distal end to the body of the catheter 10. Thus, the body 22 of the distal end 20 of the catheter 10 is inserted at least partially into the first lumen 24. Reducing the length between the perimeter of the body 22 to which the distal ends 36 of the branches 30 are fastened and the perimeter of the first lumen 24 to which the proximal ends 37 of the branches 30 are fastened results in the placement of the branches 30 in the form of an arc. To move the catheter 10 from its retracted position to its deployed position, the body 22 is slid from the distal end 20 in the opposite direction to that mentioned above, which causes the branches 30 to be arranged along the body 22, each along an axis substantially parallel to the longitudinal axis AL of the body 22.

### Structure of the Branches

Reference is made here to FIG. 6 which is a cross-section of a branch 30 of the medical device according to the invention. The branches 30 comprise a core 38 made of electrically conductive material which is surrounded by an electrically insulating sheath 39. The sheath 39 covers an external surface of the core 38. In this way, the branch is able to conduct an electrical current and its conductive part is electrically insulated from other objects in contact thereof. The electrically insulating sheath 39 comprises an opening 391 made on a face 32 of the branch 30 which is opposite the longitudinal axis AL of the body 22. This characteristic is particularly visible in FIG. 3. The electrically active zone 34 of branch 30, which forms the electrode, is located opposite the opening 391. This electrically active zone is formed by a part of the core 38 of branch 30 opposite the opening 391.

This arrangement of the branch 30 makes it possible to have a simple architecture wherein the core ensures the mechanical strength of the branch 30, while ensuring the electrical conduction necessary for the delivery of the first and second quantities of energy. The electrically insulating sheath 39, on the other hand, allows electrically insulating the conductive core 38 of objects close to the branch 30, in particular another branch 30 in the vicinity. According to this embodiment, it is also easy to place the active zone 34 where desired by cutting the opening in the desired zone. The electrode surface of the electrically active zone 34 can also be selected by making a greater or lesser opening 391 in the electrically insulating sheath 39.

Advantageously, the opening 391 is made by laser cutting the electrically insulated sheath 39. This arrangement allows quick, inexpensive and easily reproducible industrial implementation.

Advantageously, the electrically conductive core 38 is made of a flexible material capable of remaining within its elastic range of deformation as the catheter 10 moves from a deployed position to a retracted position and vice versa. This arrangement maintains the general shape initially desired for the branches 30 of the catheter 10 when it changes position. Indeed, if the conductor material of the core 38 were to come out of its elastic domain when moving from the retracted position to the deployed position, this could interfere for the latter to return to its initial retracted position, with the same geometry as that initially planned.

The electrically conductive core material 38 of branch 30 can be a shape memory material. This arrangement makes it possible to use the superelasticity characteristics of the shape memory material to allow the branches 30 to deform when moving from the retracted position to the deployed position and vice versa.

The material comprising the electrically conductive core 38 can be Nitinol. Nitinol is a metal alloy comprising nickel and titanium in almost equal proportions. Nitinol is a shape memory alloy with a superelasticity property. This Nitinol can be combined with other metals (platinum, gold, etc.) to improve its conductivity.

Alternatively or in combination with the electrically active part 34 made by an opening 391 in the sheath 39, one or more branches 30 may comprise a printed circuit 393 on an outer layer of the branch 30. The printed circuit 393 can be installed on the electrically insulating sheath 39. It may also be printed directly onto the sheath 39. This printed circuit 393 forms an electrode which is insulated from the core 38 of branch 30. FIG. 7, which is a cross-sectional view of a branch 30 comprising a printed circuit 393 shows this case.

One or more branches 30 may comprise several electrodes. For example, a branch may comprise several openings 391 in the electrically insulating sheath 39, so as to form several electrically active zones 34 on the branch 30. Similarly, a branch may comprise several printed circuits 393 each forming an electrode. It may also have one or more openings 391 on the same branch forming electrodes, as well as one or more printed circuits 393 forming electrodes. All of the electrodes mentioned hereinabove are capable of delivering the first quantity of energy or the second quantity of energy.

### Measuring Electrodes

Electrodes formed by electrically active zones 34 or by printed circuits 393 are capable of recording electrical activity. Thus, these electrodes are suitable for use as a measuring electrode to, for example, measure electrical characteristics of tissues located in the vicinity of said electrodes. This makes it possible, among other things, to facilitate the placing of the catheter 10 in the pulmonary vein before performing electroporation, by detecting the tissues to be treated. This is also used to check that isolation of the pulmonary vein has been carried out correctly after ablation.

In addition, at least one branch 30 may comprise, in addition to the electrode formed by the active zone 34 or by the printed circuit 393, one or more measuring electrodes. These measuring electrodes are preferably placed on the external surface of the branch 30. This arrangement has the advantage of using an electrode specifically designed to take measurements, enabling them to be taken with greater accuracy.

Advantageously, each branch 30 comprises at least two electrically distinct active zones 34, and each of these zones forms a conductive part of an electrical circuit of the medical device. Each circuit is independent of the other and is capable of respectively propagating a measured cardiac electrical current and a generated electrical current. “Measured electrical current” means a cardiac electrical current collected by an electrode when the latter is used as a measuring electrode. This current is then retrieved by a measuring device outside the catheter, such as an electrophysiology bay amplifier, a multimeter or an oscilloscope, this device being capable of displaying electrical data. “Generated electrical current” means an electrical current from a generator connected to the catheter 10, which flows through the electrical circuit and activates the electrodes connected to the circuit to deliver a quantity of energy. Thus, on the same branch, there is an electrical circuit enabling the delivery of a first quantity of energy and/or a second quantity of energy, and an electrical circuit which is adapted to it for the measurement of electrical data from a measuring electrode.

Advantageously, according to a given configuration, an ablation generator generates an electrical current generated at an electrode to perform electroporation of a branch and simultaneously a measuring device records the quantities of energy or the electrical levels of at least one measuring electrode of a same branch 30 or a separate branch. This energy or this level of electrical voltage or current measured by the measuring device can be compared with the measurement of the energy actually delivered by the generator in order to derive electrical conductivity or energy absorption parameters. This last measurement can be obtained using the ablation generator, which may comprise means to measure the energy or level of an electrical voltage or current delivered.

Advantageously, a branch 30 comprises at least one electrically active zone 34 forming a conductive part of a single electrical circuit of the medical device. The said electrical circuit of the medical device is designed to propagate the measured electrical current and the generated electrical current. This arrangement allows a single electrically active zone 34 to be used as an electrode for electroporation and as a measuring electrode. Advantageously, the branch 30 comprises only one active zone 34 forming a conductive part of the electrical circuit.

The body 22 of the distal portion 20 of the catheter 10 may comprise a measuring electrode 28. This measuring electrode 28 is capable of recording electrical activity. Advantageously, it records an electrical activity together with an electrode of an active portion 34 of a branch 30. Therefore, a voltage can be measured between a branch and the body 22 of the distal portion of the electrode. More advantageously, this electrode is a reference electrode. This makes it possible to measure an electrical potential at an electrode carried by an active zone 34 of a branch 30.

These provisions allow accurate measurements to be taken during an act of isolation of the pulmonary vein to improve catheter placement, particularly before performing electroporation, or to monitor the success of said electroporation.

### Insulation of the Branches

The branches 30 of the catheter 10 may each comprise two lateral edges 35, each lateral edge 35 comprising electrical insulation. Each lateral edge 35 can be advantageously made of electrical insulating material. This electrical insulation electrically insulates a branch 30 from a branch 30 located near the first branch 30. This arrangement makes it possible to avoid contact between two electrodes located on two different branches 30. Thus, bipolar electroporation can still be performed even if two branches have come closer together due to stresses applied to the catheter 10 by the walls of the atrium of the treated patient. Similarly, when the catheter 10 is in the retracted position, an active zone 34 of a branch 30 is thus electrically insulated from the active zones 34 of the branches directly in the vicinity of the latter. In this way, unipolar electroporation is facilitated, as the only activated electrode in this mode is insulated from the other electrodes of the catheter 10. A controlled electrode surface is therefore maintained and possible short circuits due to unwanted electrical contacts between several electrodes are avoided.

Advantageously, this insulation is made directly in the insulating sheath 39 of the branch 30, in the case where an opening is made only on the face opposite the longitudinal axis AL of the body 22. In this configuration, each lateral edge 35 of the branch 30 is covered by the insulating sheath 39. This arrangement makes it possible to insulate the side edges 35 of the branch 30 directly with the insulating sheath 39, which means that no additional insulating elements have to be added to the branches, thereby making it easier and less expensive to manufacture.

It can also be provided that the active zones 34 of the branches are separated from the lateral edges 35 by an insulating buffer zone 351 located on the face of the branch 30 opposite the longitudinal axis AL of the body 22 of the distal portion 20. This insulating buffer zone 351, covered with insulating material, can for example be taken from the insulating sheath 39, or an additional element fastened to the branch 20. For example, if a branch 30 is twisted, this buffer zone makes it possible to ensure that the active zone 34 of a given branch does not come into contact with another active zone of another branch in the vicinity. Advantageously, this insulating buffer zone 351 has the width of a branch 30.

The insulation of the lateral edges 35 can also be achieved by adding insulation elements made of insulating material that cover the lateral edge 35, which allows good electrical insulation of the branches 30 with respect to each other.

Alternatively or additionally, a set of splines can be provided on the body 22. These splines extend on an external surface of the body 22 in a longitudinal direction parallel to the longitudinal axis of the body 22. These splines are designed to accommodate the branches 20 in the grooves they form when the catheter is in the retracted position. Thus, in the retracted position, each branch 20 is separated from the two branches 20 in the vicinity of the latter by the portion farthest from the longitudinal axis AL of the body 22 of the spline. This part of the spline is preferably made of electrically insulating material. The advantage of this arrangement is that the different branches 20 can be electrically insulated when the catheter 10 is in the retracted position. Thus, when the catheter 10 delivers the second quantity of energy, the active zone 34 used to deliver this second quantity of energy is electrically insulated from the other branches 20 (and therefore from the other active zones 34) by the splines. Therefore, the surface of the electrode used is controlled precisely and short circuits which could deteriorate the quality of the unipolar electroporation performed are avoided.

### Arrangement of Active Zones

Each active zone 34 of each branch 30 is distant from the distal end 36 of said branch 30. Additionally, each active zone 34 of the branch 30 is distant from the proximal end of said branch 30. This arrangement allows adequate placement of the active zones for the creation of an electrical field to perform bipolar electroporation when the catheter 10 is in the deployed position.

According to one embodiment, the distal portion 20 of the catheter 10 comprises an equatorial plane PE perpendicular to the longitudinal axis AL of the body 22. The equatorial plane PE cuts the branches 30 equidistant from their proximal 37 and distal 36 ends. Each active zone 34 of the branches 30 is centered around the equatorial plane PE. This arrangement makes it possible to have the active zones 34 in the portion of the branch 30 which is farthest from the longitudinal axis AL of the body 22 when the catheter is in the deployed position. Thus, the active zones 34 are as close as possible to the walls of the pulmonary vein and of the ostium and can therefore create effectively electric fields of electroporation.

According to one embodiment, the active zone 34 is offset in the direction of the distal end 36 of the branch 30 with respect to the equatorial plane PE. This characteristic allows a better arrangement of the active zones 34 when the catheter 10, when deployed, is not “straight” in the pulmonary vein, i.e. when the longitudinal axis AL of the body 22 is not substantially parallel locally to a longitudinal axis of the pulmonary vein, at the level of the equatorial plane PE. This advantageous arrangement allows effective electroporation in the deployed position even when the catheter 10 has not deployed optimally. Advantageously, the active zone 34 of a branch 30 is offset by at least 7 millimeters from the proximal end 36 of the branch 30. A considered offset of the active zone 34 is 9 millimeters, but other offset values may be considered.

Advantageously, the active zone 34 is offset in the direction of the proximal end 37 of the branch 30 with respect to the equatorial plane PE. This characteristic allows a better arrangement of the active zones 34 when the catheter 10, when deployed, is not “straight” in the pulmonary vein, i.e. when the longitudinal axis AL of the body 22 is not substantially parallel locally to the longitudinal axis of the pulmonary vein, at the level of the equatorial plane PE. This advantageous arrangement allows effective electroporation in the deployed position even when the catheter 10 has not deployed optimally.

According to one embodiment, each branch 30 comprises an arrangement of its active zone(s) 34 similar to the arrangement of the other branches 30 of the catheter 10.

According to one embodiment of the invention, a branch 30 comprises an arrangement of its electrically active zone 34 different from the arrangement of the electrically active zone 34 of the branch or branches 30 which are located directly adjacent to the branch 30 in question. This arrangement makes it possible to have different active zone 34 layouts from one branch 30 to the other. This is particularly advantageous when the catheter 10, when deployed, is not “straight” in the pulmonary vein, i.e. when the longitudinal axis AL of the body 22 is not substantially parallel locally to the longitudinal axis of the pulmonary vein, at the level of the equatorial plane PE. This advantageous arrangement allows effective electroporation in the deployed position even when the catheter 10 has not deployed optimally.

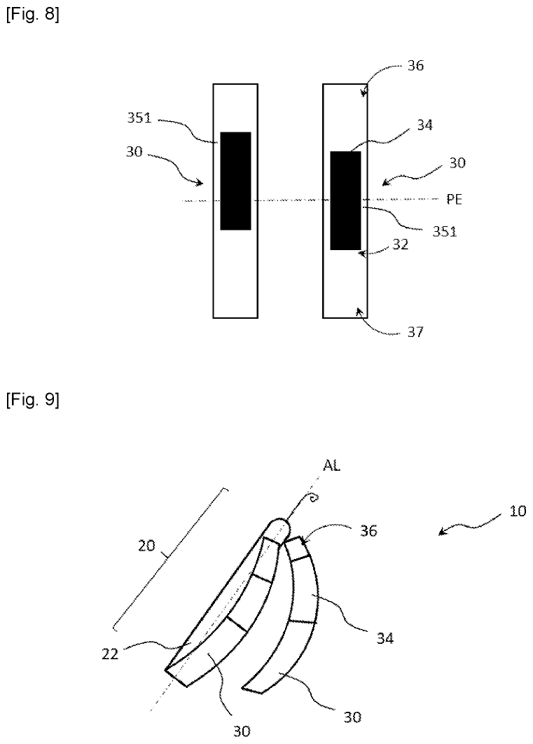

FIG. 8 shows successive branches 30 on the catheter 10 that have their active zones 34 longitudinally offset from each other. FIG. 9 shows the arrangement of these two branches 30 on the body 22 of the distal portion of the catheter 10.

Advantageously, the active zone 34 of a branch 30 is offset longitudinally with respect to the active zone of the branch located directly in the vicinity of the latter.

Advantageously, the active zone 34 of a branch 30 is longitudinally offset by 2 millimeters in the direction of the proximal end 37 with respect to the active zone 34 of the branches 30 located directly in the vicinity of the first branch 30 mentioned. This arrangement allows good creation of an electric field between two successive branches 30 along the perimeter of the body 22. This arrangement is also advantageous in the event that the catheter 10 has not deployed “straightly” into the pulmonary vein. The longitudinally offset active zone 34 may also be offset only on its end close to the distal end 36, the other end of the active zone 34, the one closer to the proximal end 37, being at the same level as that of the active zone 34 of the branch 30 located directly in the vicinity of the latter. In other terms, the active zone 34 of a branch 30 may be longer than that of the branch 30 located directly in the vicinity, and be closer to the distal end 36 of the branch 30, and at the same time be at the same distance from the proximal end 37 of the branch as the active zone 34 of the branch 30 located directly in the vicinity.

According to an embodiment of the invention, the active zone of a branch 30 is located at a distance of seven millimeters from the distal end 36 of said branch 30, and the active zone 34 of the branch 30 directly in the vicinity of the latter is offset by two millimeters longitudinally towards the proximal end relative to the first branch 30.

### Branches and Fastenings

The branches 30 have a general ribbon shape. “Ribbon shape” means that the branches 30 comprise a substantially rectangular cross-section. Thus, the faces 32 of the branches 30 opposite the longitudinal axis AL of the body 22 are substantially flat and are wider than the lateral edges 35 of the latter. The ribbon shape allows good stability of the branch when the catheter 10 is in the deployed position, with the faces 32 bearing on the walls of the pulmonary vein. This characteristic reduces unwanted twisting of the branches 30. In the retracted position, the ribbon shape allows for an arrangement in rows of the branches 30 around the body 22, which facilitates unipolar electroporation when the catheter 10 is in the retracted position. Specifically, the ribbon shape of the twigs 30 during deployment allows maintaining a regular geometry of the arrangement of the twigs 30 in relation to each other. This shape provides sufficient stiffness to each branch 30 to prevent it from twisting during deployment. This is because this characteristic prevents certain branches, once deployed, from not being evenly arranged about the longitudinal axis AL. Such an incorrect arrangement means that the distance between each branch is not always the same. In this way, a branch could be too close to a branch directly in the vicinity of it, which would lead to a risk of arcing between the two branches during electroporation. Similarly, a branch could be too far away from the next branch about the longitudinal axis AL. In this case, an excessive distance could lead to a defect in the electroporation which could be incomplete on the perimeter of the vein to be isolated. The ribbon shape of the branches 30 therefore allows the branches 30 to remain evenly and symmetrically arranged during deployment.

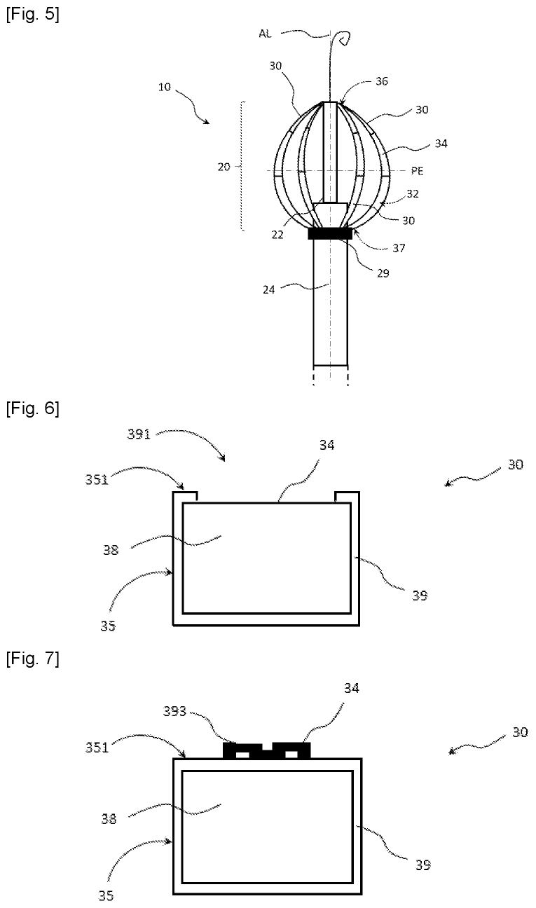

According to an embodiment shown in FIG. 5, the fastening of the proximal ends 37 of the branches 30 to the perimeter of the first lumen 24 is carried out via a fastening ring 29. This fastening ring 29 grips the outer surface of the catheter 10 slightly upstream from the distal portion 20 of the catheter 10. Advantageously, it is possible to adjust the position of the fastening ring 29 on the catheter 10. This adjustment is used to adjust the radial distance that the branches 30 will travel during deployment, in order to best adapt to the size of the pulmonary vein that will be treated.

### Guiding of the Catheter

In order to treat the patient, according to a possibility, the catheter 10 is inserted into an introducer itself inserted into a blood vessel until the atria. The catheter will navigate through the left atrium towards the pulmonary vein to isolate it. For this purpose, it is possible to provide in the body 22 of the distal portion 20 a second lumen (not shown) which is adapted to receive a guiding device, such as that shown in FIG. 1. In this example, the guiding device 40 comprises a rounded or curved distal end to avoid perforating a wall of a vessel or organ. Prior to passing through the second lumen of the body 22, the guiding device 40 is accommodated by the first lumen 24 of the catheter 10.

Advantageously, the first and/or second lumen has a diameter adapted to receive a guiding device.

Advantageously, the second lumen opens onto a distal end of the body 22.

The guiding device 40 may advantageously be a guiding catheter

The guiding device may advantageously be a guidewire. “Guidewire” means a metal guide.

Alternatively or additionally, the guiding device may comprise an internal mechanism integrated into the catheter 10. The internal mechanism may allow orientation of the distal portion 20 of the catheter 10. This internal mechanism may, for example, comprise one or more rods making it possible to bend said distal part 20 and/or to orient it. This mechanism can be activated and/or controlled from a handle or proximal control device of the catheter.

The guiding device may comprise means of driving the distal portion 20 into a blood vessel. In one case, the guiding device, when inserted into the cavity 24, may cause the longitudinal extraction of the body 22 from the catheter 10. This driving may, for example, be provided by a shoulder or a circumferential stop of the guiding device 40 which comes into contact with a portion of the body 22. It may advantageously comprise means of fastening for fastening the guiding device 40 to the distal portion 20 of the catheter 10.

According to one embodiment, the deployment of the branches 30 fastened to the body 22 is ensured by inflating a balloon. This variant allows the surface of the balloon to be used as a support for the electrodes. This embodiment allows stabilizing and ensuring proper positioning of the branches when they are deformed. According to another example, the balloon may be substituted or combined with a deformable mesh, such as a latticework mesh. For example, a shape memory material can be used to yield a stable shape of the deployed mesh in 3D.

The guiding device may comprise an articulated head to guide the catheter 10 in the blood vessels and in the heart allowing it to follow turns in said blood vessels and in the heart.

The guiding device may also guide the catheter 10 with its articulated head out of the second lumen, with the head exiting the lumen through the distal end of the shaft 22.

### Electroporation Methods

According to one embodiment, the medical device comprises, in addition to the catheter 10, a generator adapted to deliver electrical energy to the active zones 34 of branches 30.

Advantageously, the first quantity of energy is delivered according to a train of electrical pulses that are generated within at least two branches 30 when the catheter 10 is in the deployed position. These electrical pulses generate electrical voltage pulses between two active zones 34 of two branches 30. The pulses have durations of about one or a few microseconds. This arrangement is advantageous for implementing electroporation in a bipolar mode.

It may be noted that the pulse sequence may be performed by successively supplying several electrode pairs of pairs of branches 30. In each pair of branches 30 selected, the two branches 30 may be two successive branches on the perimeter of the body 22.

Advantageously, the second quantity of energy is delivered according to a train of electrical pulses that are generated within at least one branch 30 when the catheter 10 is in the retracted position. These electrical pulses generate electrical voltage pulses between the active zone 34 of a branch 30 and an external electrode that is placed on the skin of the patient, or between two active zones 34 of two branches 30. The pulses have durations of about a microsecond. This arrangement is advantageous for implementing electroporation in a unipolar or bipolar mode.

### NOMENCLATURE

10: catheter
20: distal portion of catheter
22: body of the distal portion
24: first lumen
28: body measuring electrode
29: fastening ring
30: branch
32: face of the branch
34: electrically active zone
35: lateral edge of the branch
351: insulating buffer zone
36: distal end of the branch
37: proximal end of the branch
38: core of the branch
39: insulating sheath of the branch
391: opening in the insulating sheath
393: printed circuit
40: guiding device
AL: longitudinal axis of the body
PE: equatorial plane of the distal portion

1. A medical device comprising a catheter comprising a mobile distal portion comprising a body adapted to move longitudinally in a first lumen and a plurality of branches, a distal end of each branch being fixed along a perimeter of said body and a proximal end 37 being fixed along a perimeter of said first lumen, each branch comprising a face having at least one electrically active zone forming an electrode, the branches being mobile between at least two positions that include:
a retracted position wherein the branches are each arranged along an axis parallel to the longitudinal axis of the body;
a deployed position wherein the branches each form an arc, each arc extending in a plane to which the longitudinal axis of the body belongs, the medical device being electrically configured to deliver a first quantity of electrical energy to a first set of electrodes in the deployed position and a second quantity of electrical energy to at least one electrode in the retracted position.

2. The medical device according to claim 1, wherein the body of the distal portion comprises a second lumen adapted to receive a device for guiding the catheter.

3. The medical device according to claim 2, wherein the medical device is configured to deliver the first quantity of energy or the second quantity of energy according to an electrical pulse train at at least one branch resulting in a creation of electrical voltages between two electrodes.

4. The medical device according to claim 3, wherein a first electrode is at least one first branch and a second electrode is at least one second branch.

5. The medical device according to claim 4, wherein the first quantity of electrical energy is delivered according to a sequence comprising a succession of supplies of pairs of electrodes.

6. The medical device according to claim 3, wherein a first electrode is at least one branch and a second electrode is an electrode external to the catheter.

7. The medical device according to claim 1, wherein each branch comprises a core made from an electrically conductive material surrounded by an electrically insulating sheath comprising an opening, the active zone being arranged on a face opposite a longitudinal axis of the body through said opening.

8. The medical device according to claim 1, wherein each branch is made from a shape memory material.

9. The medical device according to claim 7, wherein the conductive material is Nitinol.

10. The medical device according to claim 1, wherein at least one electrode of a branch comprises a circuit printed on an external layer of the branch.

11. The medical device according to claim 1, wherein at least one branch comprises a measuring electrode adapted to record an electrical heart activity.

12. The medical device according to claim 1, wherein at least one branch comprises several separate electrically active zones, each electrically active zone forming an electrode, the set of electrodes of the branches adapted to be configured to deliver a quantity of energy and/or being adapted to record an electrical activity.

13. The medical device according to claim 12, wherein each branch comprises at least two electrically active zones each forming a conductive portion of an electrical circuit of the medical device, each one of the electrical circuits being independent from the other in order to respectively propagate a measured electrical current and a generated electrical current.

14. The medical device according to claim 13, wherein each branch comprises at least one electrically active zone formant a conductive portion of a single electrical circuit of the medical device in order to propagate a measured electrical current or a generated electrical current.

15. The medical device according to claim 1, wherein the body of the distal portion of the catheter comprises a measuring electrode able to record an electrical activity.

16. The medical device according to claim 1, wherein each branch comprises two lateral edges, with each lateral edge comprising an electrical insulation electrically insulating said branch from a branch located in the vicinity of the latter.

17. The medical device according to claim 1, wherein each active zone of each branch is located on a portion separate from its distal end and from its proximal end, the active zone.

18. The medical device according to claim 1, wherein an active zone of the distal end of a branch is longitudinally offset from an active zone of the distal end of a consecutive branch on the perimeter of the first lumen.

19. The medical device according to claim 1, wherein the branches have a general ribbon shape.

20. The medical device according to claim 1, wherein the fixing of the branches along the perimeter of the first lumen is carried out by a fastening ring on which the proximal ends are fixed, the fastening ring itself being linked to the perimeter of the first lumen.
